(19) United States
(12) Patent Application Publication
(10) Pub. No.: US 2018/0326095 A1
(43) Pub. Date: Nov. 15, 2018

(54) KITS AND METHODS TO DETECT PENICILLIN ALLERGY

(71) Applicant: AllerQuest LLC, Plainville, CT (US)

(72) Inventors: Louis M. Mendelson, Bloomfield, CT (US); Charlotte Ressler, Farmington, CT (US); James D. Wolfe, Los Altos Hills, CA (US); N. Franklin Adkinson, Baltimore, MD (US); Richard Bauer, Monroe, CT (US); William Ryan, Monroe, CT (US)

(21) Appl. No.: 15/975,070

(22) Filed: May 9, 2018

Related U.S. Application Data
(60) Provisional application No. 62/504,687, filed on May 11, 2017.

(51) Int. Cl. A61K 49/00

(52) U.S. Cl. CPC A61K 49/0006

(57) ABSTRACT

The present invention is directed to a kit for evaluating on the skin of a patient the sensitivity to penicillin, comprising (A) a first vial containing a major determinant mixture, the major determinant mixture comprising benzylpenicilloyl polylysine; (B) a second vial containing lyophilized minor determinant mixture, the minor determinant mixture comprising a lyophilized mixture of neutralized (1) penicillin G potassium; (2) penicilloic acid; and (3) penilloic acid; (C) a third vial containing amoxicillin sodium; and (D) instructions for carrying out a method to evaluate on the skin of a patient the sensitivity to penicillin. The present invention is also directed to a method to rule out penicillin allergy using the disclosed kit.

### CROSS-REFERENCE TO RELATED APPLICATIONS

This Application claims the benefit of U.S. Provisional Application Ser. No. 62/504,687 filed May 11, 2017, which is incorporated by reference in its entirety herein.

### BACKGROUND OF THE INVENTION

### Field of the Invention

The present invention relates to diagnostic kits, and more particularly to diagnostic kits for determining the absence of penicillin allergy or hypersensitivity on the skin of a patient.

### Brief Description of the Related Art

Penicillin is a generic term for a family of antibiotics useful for treating infections caused by bacteria. Penicillin antibiotics are generally broad spectrum, working against a variety of organisms, and are the preferred drugs in many infections because their toxicity is low and their effectiveness at treating infections is high. Although overuse of various forms of penicillin in recent times has given rise to bacteria that are resistant to certain penicillin forms, many varieties of penicillin are in use today and effectively treat many bacterial infections.

About 10% of the US population reports a history of penicillin allergy, yet studies have consistently shown that 90% or more of these individuals are not allergic and able to tolerate penicillins (Fox S, Park M. J Allergy Clin Immunol Pract. (2014) 2:439-44; Macy E, Ngor E W. J Allergy Clin Immunol Pract. (2013) 1:258-63). The discrepancy between self-reported and confirmed penicillin allergy is partly due to resolution of penicillin allergy, since it is known that in most, but not all, patients penicillin-specific IgE antibodies wane over time (Blanca M, Torres M J, Garcia J J, Romano A, Mayorga C, deRamon E, et al. J Allergy Clin Immunol. (1999) 103:918-24). Additionally, some historical reactions did not represent true penicillin allergy, but rather were predictable side-effects or manifestations of the underlying illness. Patients with a history of penicillin allergy, compared with those without a history of penicillin allergy, are more likely to be treated with broad-spectrum antibiotics, such as quinolones and vancomycin, as well as with antibiotics with a less favorable side effect profile, such as clindamycin (Lee C E, Zembower T R, Fotis M A, Postelnick M J, Greenberger P A, Peterson L R, et al. Arch Intern Med. (2000) 160:2819-22; MacLaughlin E J, Saseen J J, Malone D C. Arch Fam Med. (2000) 9:722-6; Macy E, Contreras R. J Allergy Clin Immunol. (2014) 133:790-6.). Similarly, patients labeled penicillin allergic are more likely to be diagnosed with vancomycin resistant enterococcus (VRE), methicillin-resistant Staph aureus (MRSA) and Clostridium difficile (C. diff, require longer hospital stays, and have higher medical costs (MacLaughlin E J, Saseen J J, Malone D C. Arch Fam Med. (2000) 9:722-6; Macy E, Contreras R. J Allergy Clin Immunol. (2014) 133:790-6; Picard M, Begin P, Bouchard H, Cloutier J, Lacombe-Barrios J. J Allergy Clin Immunol Pract. (2013) 1:252-7). Consequently, penicillin allergy is not a benign diagnosis and its misdiagnosis is common.

Moreover, the scope of the problems related to a diagnosis of penicillin allergy are quite substantial when one considers that approximately 27 million (or 90%) of 30 million Americans who self-report penicillin allergy are mislabeled as penicillin-allergic. Recently, the US Center for Disease Control and Prevention (CDC), the National Quality Forum, and the American Academy of Allergy, Asthma and Immunology have highlighted that penicillin allergy is a serious public health problem and that penicillin skin testing should be an integral part of a comprehensive antibiotic stewardship program to help combat this antimicrobial resistance epidemic.

Penicillin skin testing is a validated method to rule out IgE-mediated penicillin allergy. Negative penicillin skin tests is desirable as they allow for expansion of antibiotic treatment choices for patients and their physicians. Hence, removing an erroneous penicillin allergy label from numerous patients could play an important role in antibiotic stewardship by shifting antimicrobial treatment from quinolones, vancomycin and clindamycin to less problematic, more appropriate choices.

Various skin tests for penicillin allergy or hypersensitivity have been described in the literature. For example, see Adkinson N F, Jr, Thompson W L, Maddrey W E, Lichtenstein L M; Routine use of penicillin skin testing on an inpatient service N. Engl. J. Med. (1971) 285:22-24. Sullivan et al. J Allergy Clin. Immunol. (1981) 68:171-180 describes a skin test to detect penicillin allergy in patients by testing with penicilloyl-poly-L-lysine, benzylpenicillin G, or benzylpenicilloic acid. In addition, U.S. Pat. No. 3,867,365 (1975) and 3,979,508 (1976) assigned to Kremers Urban Company disclose penicilloyl-polylysine conjugates which are useful for eliciting cutaneous responses in persons with penicillin hypersensitivity. U.S. Pat. Nos. 4,183,910; 4,228,147; 4,252,784; and U.S. Pat. No. 4,316,882 to Levine in the early 1980s disclose various other derivatives of penicilloyl-polylysine for use as components in minor determinant mixtures for skin tests to predict and diagnose penicillin allergy or hypersensitivity in a patient.

European Patent Application EP 0367090 (1994) to Schwarz Pharma AG discloses a storage-stable, lyophilized minor determinant mixture (MDM) composition comprised of benzylpenilloate, benzylpenicillin, and benzylpenicilloate which can be reconstituted with water and which is used to detect allergy or hypersensitivity to penicillins. See also Nolan et al. (2008), Internal Medicine Journal 38:357.

A commercial kit for penicillin allergy testing is sold by Diater Laboratorio de Diagnostico y Aplicaciones Terapeuticas, S.A. (Madrid, Spain) and includes a first vial containing benzypenicilloyl octa-L-lysine as the active substance, a second vial containing sodium benzylpenilloate as the active substance, and a third vial containing a phosphate buffer. The kit is marketed and sold for the diagnostic assessment of allergic, sensitization, or type I hypersensitivity conditions, in those cases where an allergy to beta-lactam antibiotics is suspected. See, for example Nolan R C, Puy R, Deckert K, O'Hehir R E, Douglass J A. Internal Medicine Journal. (2008) 38:357-367.

While the above prior art compositions and methods may be useful as individual components in a skin test for allergic reactions or hypersensitivity to penicillin, penicillin skin testing is grossly underutilized, due in large measure to the lack of the commercial availability of a skin testing kit that provides a comprehensive diagnosis. What is needed in the art is a comprehensive, standardized single-patient kit and method to assure patients are not incorrectly labeled as penicillin-allergic. The present invention is believed to be an answer to that need.

### SUMMARY OF THE INVENTION

In one aspect, the present invention is directed to a kit for evaluating on the skin of a patient the sensitivity to penicillin, comprising:

(A) a first vial containing a major determinant mixture, the major determinant mixture comprising benzylpenicilloyl polylysine;

(B) a second vial containing lyophilized minor determinant mixture, the minor determinant mixture comprising a lyophilized mixture of neutralized:
(1) penicillin G potassium;
(2) penicilloic acid; and
(3) penilloic acid;

(C) a third vial containing amoxicillin sodium; and

(D) instructions for carrying out a method to evaluate the sensitivity to penicillin on the skin of a patient.

In another aspect, the present invention is directed to a method of evaluating the sensitivity to penicillin on the skin of a patient using the above kit.

### DETAILED DESCRIPTION OF THE INVENTION

After the immunochemistry of penicillin was elucidated in the 1960s, penicillin skin test reagents were developed, but to date implementation of rapid and comprehensive penicillin skin tests has faced a number of technological obstacles. It is known that penicillin spontaneously breaks down to form a number of active moieties referred to as the major allergenic determinant (penicilloyl) and several minor allergenic determinants (the most important of which are penicilloate and penilloate). Penicillin G (also known as benzylpenicillin) and amoxicillin are also considered minor determinants. Individually, these determinants have been shown to be useful in penicillin allergy determinations, but their synthesis, access, and practical applications in the form of test kits has to date proven difficult. For example, penicillin G needs to be diluted to 10,000 U/ml for skin testing and is used off-label as the only available minor determinant in the US. Penicilloate and penilloate must be synthesized and lyophilized for use in skin testing. In the US, amoxicillin is available only in oral, not intravenous (IV) formulations, and therefore cannot be used for skin testing. In addition to barriers to access appropriate penicillin skin test reagents, IgE to minor determinants has been associated with a particularly high risk of anaphylaxis (Levine B B, Redmond A P. Intl. Arch. Allergy Appl. Immunol. (1969) 35 (5) 445-455).

While individually each of the above determinants has been used for penicillin allergy determinations, use of each determinant individually in penicillin allergy testing does not conclusively prove that a particular person is or is not allergic to penicillin because each of the above determinants omits a significant portion of patients with penicillin allergy. For example, when testing for penicillin allergy using only benzylpenicilloyl polylysine (sold and known commercially as “PRE-PEN”®), approximately 25-40% of patients with penicillin allergy are not identified. Thus, approaches to better diagnose penicillin allergy with higher reliability and increased negative predictive value would be desirable.

The present invention is a single patient use diagnostic kit containing 3 skin test reagents (benzylpenicilloyl polylysine, a minor determinant mixture, and amoxicillin) and optional ancillary supplies and reagents to detect IgE sensitization to penicillin antigens. The test reagents are contained in vials, ampoules, or other suitable containers known and appreciated by those of skill in the art. The kit offers advantages over previous individual diagnostic reagents and will reliably rule out the potential for immediate life threatening penicillin allergic reactions with a high degree of probability in patients with histories of IgE-dependent penicillin allergy.

As described above, the present invention is directed to a kit for evaluating on the skin of a patient the sensitivity to penicillin, and a method of using the kit to evaluate penicillin sensitivity on the skin of a patient by determination of IgE mediated response. The kit of the present invention includes the following as main components:

(A) a first vial (or ampoule) containing a major determinant mixture, the major determinant mixture comprising benzylpenicilloyl polylysine;

(B) a second vial containing lyophilized minor determinant mixture, the minor determinant mixture comprising a lyophilized mixture of neutralized:
(1) penicillin G potassium;
(2) penicilloic acid; and
(3) penilloic acid;

(C) a third vial containing amoxicillin sodium; and

(D) instructions for carrying out a method to evaluate the sensitivity to penicillin on the skin of a patient. Each of these components are described in more detail below.

As outlined above, the kit of the present invention includes a first vial or ampoule that contains a major determinant mixture that includes benzylpenicilloyl polylysine as the primary ingredient. Benzylpenicilloyl polylysine is available commercially under the trade name “PRE-PEN®” (manufactured by AllerQuest LLC, Plainville, Conn., and distributed by ALKAbello, Inc., Round Rock, Tex.). PRE-PEN is a synthetic skin test antigen reagent that reacts specifically with benzylpenicilloyl IgE antibodies to initiate the release of chemical mediators which produce an immediate wheal and flare reaction at a skin test site. All individuals exhibiting a positive skin test to PRE-PEN possess IgE against the benzylpenicilloyl structural group which is a hapten. A hapten is a low molecular weight chemical that conjugates with a carrier (e.g. poly-1-lysine) resulting in the formation of an antigen with the hapten's specificity. The benzylpenicilloyl hapten is the major antigenic determinant in penicillin allergic individuals. However, some individuals reacting positively to PRE-PEN will not develop a systemic allergic reaction on subsequent exposure to therapeutic penicillin, especially among those who have not reacted to penicillins in the past. Thus, the PRE-PEN skin test determines the presence of penicilloyl IgE antibodies which are necessary but not sufficient for acute allergic reactions due to the major penicilloyl determinant.

PRE-PEN® (benzylpenicilloyl polylysine injection USP) is available as a sterile solution of benzylpenicilloyl polylysine in a concentration ranging from about 1×10⁻⁵ M to about 10×10⁻⁵ M, and preferably about 6×10⁻⁵ M (benzylpenicilloyl) in a buffer, for example 0.01 M phosphate buffer and 0.15 M sodium chloride. The benzylpenicilloyl polylysine in PRE-PEN is a synthetic derivative of poly-L-lysine, where the epsilon amino groups are substituted with benzylpenicilloyl groups (50-70%) forming benzylpenicilloyl alpha amide. The procedure for manufacturing PRE-PEN is known (see U.S. Pat. No. 3,867,365, Feb. 18, 1975). PRE-PEN has the chemical structure shown in Formula (I):

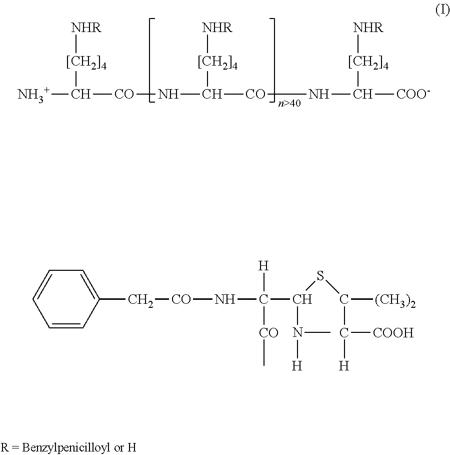

Benzylpenicilloyl polylysine is available commercially in ampoules containing 0.25 mL of solution. Ampoules are opened by snapping the neck of the vial using two forefingers of each hand. The recommended storage is refrigerated conditions (2°−8° C.). It is recommended that test antigen subjected to ambient temperatures for more than 24 hours be discarded.

The kit of the present invention includes a second vial or ampoule that contains a minor determinant mixture (MDM). This minor determinant mixture comprises a lyophilized mixture of neutralized penicillin G potassium (benzylpenicillin), penilloic acid (benzylpenilloic acid), and penicilloic acid (benzylpenicilloic acid), such as that disclosed in European Patent Application EP0367090, incorporated by reference herein in its entirety. In one embodiment, the MDM is in the form of a white, lyophilized powder cake consisting of the three active ingredients: benzyl-D-penilloic acid hydrate, benzyl-D-penicilloic acid hydrate, and penicillin G potassium, as well as a sodium phosphate buffer. The material product is preferably packaged in a 2 mL glass vial, with a stopper and an aluminum crimp seal with blue top. Useful amounts of the lyophilized material contained in each vial ranges, for example, from 0.01 to 10 mg for each component, and more preferably from 0.1 mg to 5 mg for each component. In one embodiment, each vial contains 1.3 mg of benzyl-D-penilloic acid hydrate (penilloic acid hydrate), 1.5 mg of benzyl-D-penicilloic acid hydrate (penicilloic acid hydrate) and 1.5 mg of penicillin G potassium (benzylpenicillin). Each vial also contains a buffer, for example sodium monobasic phosphate hydrate. The recommended storage is refrigerated conditions (2-8° C.). In preparation for use, the lyophilized powder is reconstituted with a suitable diluent, for example, 0.4 mL of water for injection and results in a 10 mmol/L solution of each component. The reconstituted product should preferably be used within 90 minutes.

Penicillin G potassium has the chemical structure shown in Formula (II):

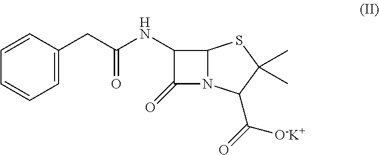

Penicilloic acid hydrate has the chemical structure shown in Formula (III):

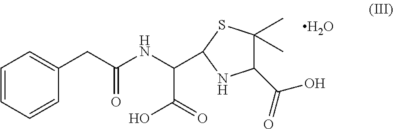

Penilloic acid hydrate has the chemical structure shown in Formula (IV):

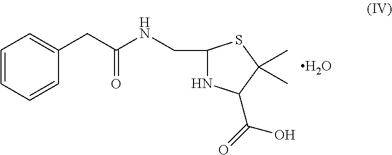

Preferably, the minor determinant mixture in the kit of the present invention is in the form of a sterile powder, and each component is present in a molar ratio of 1:1:1. In one embodiment, the second vial of the kit of the present invention preferably contains reconstituted solution of benzylpenilloic acid, benzylpenicillin, and benzylpenicilloic acid at a concentration ranging from about 0.002 M to about 0.02 M. In another embodiment, the vial contains a reconstituted solution of benzylpenilloic acid, benzylpenicillin, and benzylpenicilloic acid at a concentration ranging of about 0.001 M to about 0.01 M. In one preferred embodiment, the molar concentration of MDM for injection is about 0.01 M or about 10 mmol/L of each component. In order to maintain long shelf life, the minor determinant mixture included in the second vial of the present invention preferably has a low moisture content, desirably in the range of 1-1.5% by weight relative to the average weight of the mixture. Stabilizers, buffers, colorants, and other ingredients may also be included. In one embodiment, the vial includes a buffer, for example a sodium phosphate as buffer. The process of preparing MDM is known (Levine B B, Redmond A P. Intl. Arch. Allergy Appl. Immunol. (1969) 35 (5) 445-455; Ressler C, Neag P M, Mendelson, L M M. J Pharm Sci. (1985) 74 (4) 448-454).

The kit of the present invention includes a third vial or ampoule that contains sterilized amoxicillin sodium. Preferably, the amoxicillin sodium is in powder form, and suitable for reconstitution in water or other selected solvent. The amoxicillin sodium used in the kit of the present invention has the chemical structure shown in Formula (V):

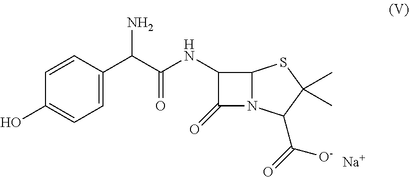 [SMILES: CC1(C)SC2C(NC(=O)C(N)c3ccc(O)cc3)C(=O)N2C1C(=O)[O-] ; [Na+]]

In the kit of the present invention, amoxicillin sodium is supplied as an off-white powder in a 2 mL glass vial containing from 1 to 50 mg of amoxicillin sodium. In one embodiment, the glass vial contains approximately 20 mg of amoxicillin sodium. Methods of manufacturing amoxicillin sodium are known in the art (see, for example, US Patent Application Publication US2002/0137926; U.S. Pat. No. 5,559,241; European Patent No. EP 0131147B2, all incorporated by reference herein). The recommended storage is refrigerated conditions 2-8° C. Preferably, the amoxicillin sodium is reconstituted with a suitable diluent, for example a saline diluent, and used within 90 minutes after reconstitution.

Without being bound by any particular theory, benzylpenicilloyl-polylysine is believed to react specifically with benzylpenicilloyl IgE antibodies to initiate release of chemical mediators which produce an immediate wheal and flare reaction at a skin test site. Historically, the benzylpenicilloyl hapten is the major antigenic determinant in penicillin-allergic individuals. The components of MDM reconstituted (benylpenicillin, benzylpenicilloate, benzylpenilloate) and amoxicillin sodium are also potential haptens which can specifically react to IgE antibodies against these forms of penicillin. These are designated as minor determinants, because they represent only 5% of the haptens, where benzylpenicilloyl represents 95%. IgE antibodies to the minor determinants may nevertheless be associated with significant clinical hypersensitivity. Together, identification of these haptens by the combination of components in the kit of the present invention provide for greater accuracy in the determination of penicillin-sensitive individuals.

The kit of the present invention further includes one or more vials or ampoules of diluents for reconstitution of the powder components (e.g., amoxicillin component and the MDM component). The diluents may be any suitable diluent, such as sterilized water or a sterilized saline solution. The volume of the diluents in each vial can be any volume suitable for use in the kit, for example 0.5 to 1.0 ml per vial. In one preferred embodiment, sterile water for injection is included in a vial in the kit for use as a diluent in reconstituting a solution of MDM. In another embodiment, a sterile solution of normal saline is included in a vial in the kit for use as a diluent in reconstituting a solution of amoxicillin sodium. It will be appreciated by those skilled in the art that upon reconstitution of solid, dry materials in the acid form with the said diluents, those dry, solid materials are solubilized to their respective anionic or cationic forms in solution. It will also be appreciated by those skilled in the art that other diluents, such as alcohols, sugars, or other compounds known in the art may be used as suitable diluents in the kit of the present invention.

The kit of the present invention may also include vials or ampoules that contain control materials. Such materials include both negative and positive controls, and when used in conjunction with the kit and method of the present invention, serve to establish a baseline to compare the results of main components of the kit (PRE-PEN, amoxicillin sodium, and MDM). In one embodiment, a suitable negative control includes sterile normal saline. In another embodiment, a suitable positive control includes histamine, morphine, codeine and the like. Other positive controls are known to those of skill in the art.

The kit of the present invention may also include optional additional components that are used to implement the testing methodology and interpret the results. For example, the kit of the present invention may also include skin test applicators for the administration of the test materials to the skin (epicutaneous). Examples of such applicators are known to those skilled in the art and include syringes, needles, single and multitest bifurcated needles, and the like. Test templates for marking the test sites and tracking administration of the test reagents and controls are also known to those skilled in the art and may also be included in the kit of the present invention. In order to interpret the results of the tests, one or more skin test rulers may be included in the kit of the present invention. Alcohol wipes may also be included to assure proper aseptic practice in implementing the kit and method of the invention. Additional components such as those described above are well known in the art of skin and allergy testing may also be in the kit of the present invention.

The carton also includes an insert that includes instructions on how to use the components and implement the method of the kit.

The above components of the kit of the present invention, including the main components, controls, instruction sheet, and optional components, are preferably housed in a carton made from plastic, cardboard, or other suitable material. Preferably, the components of the kit are arranged in the carton in a layout that promotes efficiency in implementing the tests, while simultaneously minimizing the chance of confusion between selections of active components. The carton may also be light-blocking, mold resistant, and safety sealed in order to maintain the freshness and integrity the components stored inside. Preferably, all the components of the kit are made for a single patient use.

The kit of the present invention offers significant advantages in penicillin allergy diagnostics as compared to use of each of the above determinants individually. As mentioned above, when testing for penicillin allergy using only benzylpenicilloyl polylysine (PRE-PEN), approximately 25-40% of patients with penicillin allergy are not identified. However, by following with a second diagnostic procedure where penicillin G potassium is used as the determinant, the population of unidentified patients with penicillin allergy reduces to approximately 5%. Adding a third diagnostic procedure using the remaining determinants of the present invention further reduces the unidentified population to 2-3%. Thus, the specific combination of components and determinants of the kit of the present invention provide synergies in the diagnosis of penicillin allergies in patients that increases the negative predictive value of the results. This translates into higher confidence on the part of medical and health professionals that a particular patient does or does not have a penicillin allergy. As would be appreciated by one skilled in the art, such synergies would not be present by utilizing the components of the present kit individually to diagnose penicillin allergy.

### EXAMPLES

The following examples illustrate the negative predictive value of penicillin skin testing utilizing skin test reagents (benzylpenicilloyl polylysine, penicillin G potassium, penicilloate, penilloate, and amoxicillin sodium) produced to FDA requirements, specifically for skin-testing use in a group of patients with convincing histories of penicillin-induced IgE-mediated type allergy.

### Example 1

This example illustrates a prospective, open label investigation of penicillin skin testing using the kit of the present invention in subjects 18 years of age or older, with a self-reported history consistent with an IgE-mediated reaction to a penicillin class compound (defined as one or more of anaphylaxis, decreased blood pressure and/or diminished consciousness, upper or lower airway obstruction, angioedema, urticaria, and/or generalized pruritic rash). Exclusion criteria included pregnant or lactating women; penicillin reaction within 6 weeks prior to skin testing; respiratory infection or use of systemic antibiotics within 2 weeks of skin testing; use of oral antihistamines within 72 hours prior to skin testing; use of doxepin 7 days prior to skin testing; use of any new prescription medications, over-the-counter medications or herbal supplements during the 72 hours following an oral amoxicillin challenge; and individuals who had tolerated a penicillin antibiotic subsequent to their previous allergic penicillin reaction.

The penicillin skin test kit was composed of the following components in three separate vials: (1) benzylpenicilloyl polylysine (also known as penicilloyl-polylysine), 60 μmol/L, (2) a minor determinant tripartite lyophilized powder mixture (MDM) consisting of penicillin G potassium (also known as penicillin G), penicilloic acid (also known as penicilloate) and penilloic acid (also known as penilloate), each component ranging from 1 to 25 mg per vial, and preferably approximately 10 mg per vial, and (3) amoxicillin (amoxicillin sodium) powder, 20 mg/mL. Skin test reagents were synthesized and checked for purity and stability according to FDA requirements.

The primary endpoint was negative predictive value (NPV), which was estimated as p=percentage of n history-positive subjects with negative skin tests who did not experience an IgE-dependent hypersensitivity reaction within 72 hours of a single 250 mg oral amoxicillin trihydrate challenge. Uncertainty in the estimated NPV was indicated as exact 95% confidence interval (CI) based on the binomial distribution and calculated using statistical analysis software (SAS). The primary analysis was a z-test of the null hypothesis that the NPV was inferior to 94%, the lower 95% CI for three similar published studies. The determination of study sample size was based on a test of non-inferiority with published similar studies with a power of 0.8 so that the NPV was not less than 94%. Assuming that 15% of screened subjects would be skin test-positive (and ineligible for amoxicillin challenge), an estimated total study size of 435 was required to achieve the target sample size of 370 history-positive, skin test-negative subjects to undergo amoxicillin challenge and subsequent 72-hour assessment of outcome. These patients were enrolled at 13 allergy centers in the US. In addition, each site evaluated two control subjects (patients with no history of penicillin allergy) to provide data on skin test specificity; these subjects did not undergo amoxicillin challenge.

Puncture (epicutaneous) skin testing was performed on the inner volar aspect of the forearm, using each allergen (benzylpenicilloyl polylysine, MDM, and amoxicillin sodium), as well as histamine and saline controls. A positive response was defined as development of a wheal measuring >4 mm in its longest diameter 15-20 minutes after application. If the puncture test was positive, intradermal skin testing was not performed using that allergen. If the puncture test was either negative or equivocally positive (<5 mm longest wheal diameter), intradermal testing was performed by injecting in duplicate an amount sufficient to raise a small intradermal bleb of about 3 mm in diameter. The skin test sites were read at 15-20 min. A negative response was defined as no increase in wheal size compared to the negative saline control site. A positive response was defined as an increase in wheal size of ≥3 mm compared to the negative saline control.

All subjects found to be skin test-negative on both puncture and intradermal skin testing to each of the reagents underwent non-blinded, single dose oral challenge with 250 mg amoxicillin trihydrate, followed by a one-hour observation period. Subjects were instructed to report any subsequent adverse reactions and were contacted 72 hours later by telephone. Adverse events (AEs) were collected after skin testing and for 3 days following oral challenge, and they were classified as IgE-dependent, non-IgE-dependent, or recognized side effect of amoxicillin therapy (e.g., gastrointestinal upset). The severity of all AEs was divided into mild, moderate, severe, life-threatening and fatal. The causal relationship to skin testing or amoxicillin challenge was separated into the following categories: unrelated, unlikely related, possibly related, probably related, and definitely related.

### Results

The total population included 481 subjects, comprised of 455 patients with a history of penicillin allergy and 26 control subjects (2 per site) without a history of penicillin allergy, all of whom were skin test-negative to benzylpenicilloyl polylysine, MDM and amoxicillin sodium reagents. The penicillin allergy history-positive subjects ranged in age from 18-87, with a mean of about 50 years (Table 1). The most common historical reaction symptoms were urticaria (59%), pruritus/flushing (53%) and macropapular rash (43%).

TABLE 1
Demographics, asthma status and penicillin allergy history of subjects
with a history of penicillin allergy by skin test result

| | Total with | | Skin Test* | | Negative | | |
|---|---|---|---|---|---|---|---|
| | Skin Test* | | Positive | | (ITT)* | | |
| Characteristics at enrollment | (N = 455) | | (N = 63) | | (N = 391) | | P† |
| Demographics | | | | | | | |
| Age (years), mean (SD) | 50.6 | (16.3) | 49.9 | (15.5) | 50.7 | (16.4) | 0.71 |
| [Minimum, maximum] | [18.4, 87.6] | | [21.9, 85.0) | | [18.4, 87.6] | | |
| Female sex | 326 | (72%) | 39 | (62%) | 286 | (73%) | 0.07 |
| Ethnicity: Hispanic | 13 | (3%) | 1 | (2%) | 12 | (3%) | 1.00 |
| Race/ethnicity | | | | | | | 0.67 |
| Any Hispanic or Latino | 13 | (3%) | 1 | (2%) | 12 | (3%) | |
| Non-Hispanic: | | | | | | | |
| White | 415 | (91%) | 58 | (92%) | 356 | (91%) | |
| Black | 13 | (3%) | 3 | (5%) | 10 | (3%) | |
| Other‡ | 14 | (3%) | 1 | (2%) | 13 | (3%) | |
| Asthma status | | | | | | | |
| Active asthma | 126 | (28%) | 17 | (27%) | 109 | (28%) | 1.00 |
| Peak expiratory flow rate | 429.5 | (93.8) | 428.2 | (59.8) | 429.7 | (98.2) | 0.93 |
| (PEFR) (L/min), mean (SD) § | | | | | | | |
| Penicillin allergy history (self- | | | | | | | |
| report) | | | | | | | |
| Penicillin compounds | | | | | | | 0.04 |
| responsible for reaction¶ | | | | | | | |
| Benzylpenicillin (Pen G or VK) | 146 | (32%) | 16 | (25%) | 130 | (33%) | |
| Amoxicillin | 123 | (27%) | 13 | (21%) | 109 | (28%) | |
| Amoxicillin/clavulanic acid | 15 | (3%) | 0 | (0%) | 15 | (4%) | |
| Other penicillin product | 9 | (2%) | 2 | (3%) | 7 | (2%) | |
| Unknown penicillin product | 162 | (36%) | 32 | (51%) | 130 | (33%) | |
| Allergy symptoms reported∥ | | | | | | | |
| Urticaria | 267 | (59%) | 38 | (60%) | 228 | (58%) | 0.78 |
| Pruritus or flushing | 242 | (53%) | 26 | (41%) | 216 | (55%) | 0.04 |
| Maculopapular rash | 196 | (43%) | 23 | (37%) | 173 | (44%) | 0.22 |
| Angioedema | 89 | (20%) | 11 | (17%) | 78 | (20%) | 0.73 |
| Shortness of breath | 54 | (12%) | 9 | (14%) | 44 | (11%) | 0.52 |
| Lightheadedness or loss of | 34 | (7%) | 5 | (8%) | 29 | (7%) | 0.80 |
| consciousness | | | | | | | |
| Nausea or vomiting or | 25 | (5%) | 4 | (6%) | 21 | (5%) | 0.77 |
| abdominal pain | | | | | | | |
| Rapid heart rate | 27 | (6%) | 4 | (6%) | 23 | (6%) | 0.78 |
| Fever | 18 | (4%) | 0 | (0%) | 18 | (5%) | 0.15 |
| Diarrhea | 8 | (2%) | 2 | (3%) | 6 | (2%) | 0.31 |
| Joint pain | 6 | (1%) | 1 | (2%) | 5 | (1%) | 0.59 |
| Treatments for reaction** | | | | | | | |
| None | 108 | (24%) | 13 | (21%) | 95 | (24%) | 0.63 |
| Antihistamine | 113 | (25%) | 18 | (29%) | 94 | (24%) | 0.43 |
| Epinephrine | 18 | (4%) | 1 | (2%) | 16 | (4%) | 0.49 |
| Corticosteroids | 34 | (7%) | 3 | (5%) | 31 | (8%) | 0.60 |
| Other | 41 | (9%) | 8 | (13%) | 33 | (8%) | 0.34 |
| Cannot recall | 185 | (41%) | 26 | (41%) | 159 | (41%) | 1.00 |
| Reaction observed by provider | | | | | | | 0.11 |
| Yes | 303 | (67%) | 37 | (59%) | 265 | (68%) | |
| No | 85 | (19%) | 18 | (29%) | 67 | (17%) | |
| Unknown | 67 | (15%) | 8 | (13%) | 59 | (15%) | |
| Assessment of IgE-mediated | | | | | | | 0.49 |
| penicillin allergy by history | | | | | | | |
| Unlikely | 5 | (1%) | 0 | (0%) | 5 | (1%) | |
| Possible | 187 | (41%) | 27 | (43%) | 160 | (41%) | |
| Probable | 224 | (49%) | 28 | (44%) | 195 | (50%) | |
| Definite | 39 | (9%) | 8 | (13%) | 31 | (8%) | |

*Table reports characteristics for the 455 total penicillin history-positive participants. Comparisons of characteristics by skin-test reported for 454 subjects, of which the 63 subjects were excluded from the amoxicillin challenge due to positive skin test results, and of which the remaining 391 skin test negative subjects comprise the Intent-to-treat (ITT) population. One negative skin-test subject included in the total column was excluded from the ITT population due to amoxicillin-challenge refusal.
No. (percent) reported for categorical characteristics; mean (standard deviation) reported for continuous characteristics.
†P determined from Fisher's exact test for categorical variables and two-sample t-tests for continuous variables.
‡Other race includes: Positive challenge (1 subject is Asian); Negative challenge (2 American Indian, 5 Asian, 1 Hawaiian/South Pacific Islander, 5 Other including mixed).
§ Peak expiratory flow rate is only collected for active asthma subject.
¶Penicillin compound reported for the subject's first reported reaction.
22/454 (5%) subjects reported 2 penicillin reactions in the history [7/63 (11%) Positive Challenge vs 15/391 (4%) Negative Challenge; P = 0.02)].
∥Subjects may report more than one symptom for the reaction; average number symptoms reported per subject (SD) = 2.1 (1.3) [min: 0, max: 9].
**Subject may report more than one treatment for the reaction.
Other treatments for reaction included: lotions, creams, ER/hospitalization, oatmeal bath, oxygen.

Table 2 summarizes the skin test patterns in the 63 subjects who had one or more positive tests. This group comprised 13.8% of history-positive subjects who were skin tested. Among the 63 skin test-positive patients, 30 (47.6%) reacted to a single reagent, and of these, MDM was positive in 80% (24/30). Only 4/63 skin test positive patients (6.3%) reacted to amoxicillin alone. PPL was positive in 34.9% of responders, MDM in 90.5%, and amoxicillin in 50.8%. 23.8% reacted to all 3 skin test reagents.

TABLE 2
Skin-Test Reagent response for the 63 subjects with positive Skin Test (ST) results

| | | Percent Positive Skin Test Patterns | |
|---|---|---|---|
| | | Among Subjects | |
| | Positive ST* | With + Skin Test | With H/O Pen allergy† |
| | (N = 63) | (N = 63) | (N = 455) |
| Skin Test Reagent | No. subjects | % | % |
| PRE-PEN only | 2 | 3.1% | 0.4% |
| MDM only | 24 | 38.1% | 5.3% |
| Amoxicillin only | 4 | 6.3% | 0.9% |
| PRE-PEN + MDM | 5 | 7.9% | 1.1% |
| PRE-PEN + Amoxicillin | 0 | 0.0% | 0.0% |
| MDM + Amoxicillin | 13 | 20.6% | 2.9% |
| PRE-PEN + MDM + Amoxicillin | 15 | 23.8% | 3.3% |

*One or more skin test reagents was positive on initial skin testing.
†H/O Pen allergy denotes “history of penicillin allergy.”

Seven of the 455 skin tested subjects had an adverse event judged probably or definitely related to penicillin skin testing. Two cases were in patients with strongly positive puncture skin tests, producing cutaneous and pulmonary reactions treated with epinephrine in one case and an antihistamine in the other. Both resolved completely with no sequalae. A third case involved a skin test site rash delayed 5 hours after application of skin testing. The remaining 4 adverse events were judged non-IgE dependent local skin test site reactions which were delayed in onset, described as hematoma, bruising or erythema.

The intention to treat (ITT) group consisted of all 391 history-positive, penicillin skin test-negative subjects, each of whom underwent oral challenge with 250 mg amoxicillin. Eighteen patients from the ITT population were excluded from the per protocol (PP) group (N=373), for exclusionary histories as follows: 3 individuals tolerated a penicillin compound after their initial reaction; 5 subjects previously had an unlikely IgE-dependent penicillin reaction; and 10 patients had a history of generalized rash only, but without data on the onset time of symptoms (Table 3).

TABLE 3
Negative Predictive Value (NPV) of Skin Testing with
PRE-PEN Skin Test: Primary and secondary outcomes

| | IgE-mediated reaction* | | NPV | |
|---|---|---|---|---|
| | Yes† | No | (95% C.I.)‡ | P‡ |
| Primary outcome | | | | |
| Intention-to-treat§ | 8 | 383 | 98.0% (96.0%, 99.1%) | <0.0001 |
| Secondary outcome | | | | |
| Per-Protocol¶ | 8 | 365 | 97.9% (95.8%, 99.1%) | <0.0001 |

*A positive reaction is defined as a subject who has an IgE-dependent hypersensitivity reaction within 72 hours of the oral amoxicillin challenge, after all intradermal testing with reagents in the skin test kit were negative.
†The 8 IgE-mediated reactions were classified as: one subject with a Grade 3-severe (Definitely related to Amoxicillin), three with Grade 2-moderate (1 Probably, 2 Possibly related), and four with Grade 1-mild (all Probably related) reactions as coded using the MedDRA 16.0 classification system. There were NO serious adverse events (SAE).
The symptoms for all subjects included one or more of the following: rashes, pruritus, hives, swelling, flushing of face/scalp. The subject with severe symptoms additionally had chills, headache, vomiting, and abdominal pain (see Table 3 for detailed listing).
‡NPV denotes negative predictive value defined as the percentage of subjects in the study population with no IgE reaction. Exact 95% confidence intervals (C.I.) are calculated based on the binomial distribution.
P is a one-sided exact P value for non-inferiority; based on an equivalence test for the binomial proportion 97%, and an equivalence test margin of 3%. For the ITT analysis, Z = 5.5227; Z = 5.1395 for the PP analysis.
§The Intention-to-treat population included all subjects who had valid skin testing performed and who received the oral amoxicillin challenge; total N = 391.
¶The Per-Protocol population included all subjects in the ITT population who completed the 72+ hour telephone follow-up assessment and who did not have any major protocol deviations; total N = 373.

Following amoxicillin challenge, eight patients experienced IgE-dependent reactions that were possibly, probably or definitely related to the drug (Table 4).

TABLE 4
IgE-dependent reactions following amoxicillin challenge in penicillin history-positive subjects with negative penicillin skin tests

| Subject | | | Historical Penicillin Reaction | | | Current Study IgE Reactions to Amoxicillin | | |
|---|---|---|---|---|---|---|---|---|
| | Age | | | | AE | | | AE† |
| Sex | (yrs) | Race | Compound | Symptoms | Grade* | AE Description | Treatment for AE | Relatedness |
| M | 59 | White | Amoxicillin - oral | Urticaria, angioedema, | 3 | Abdominal pain upper, | Diphenhydramine, | Definitely |
| | | | route | pruritus, rash, lightheaded, | | generalized pruritus, pain, | prednisone, | |
| | | | | shortness of breath, fever, | | flushing after 8 hours; | inhaled albuterol | |
| | | | | rapid heart rate | | respiratory tract | | |
| | | | | | | congestions (chest), | | |
| | | | | | | chills, headache and | | |
| | | | | | | vomiting after <24 hours | | |
| F | 72 | White | Benzylpenicillin | Rash | 2 | Erythema, rash, | Cetirizine, | Probably |
| | | | (Pen G or V) - | | | angioedema: | diphenhydramine | |
| | | | intramuscular route | | | Erythematous rash and | | |
| | | | | | | facial swelling after 19 | | |
| | | | | | | hours | | |
| M | 50 | White | Benzylpenicillin | Urticaria, angioedema, | 2 | Oedema peripheral: Wrist | Diphenhydramine | Possibly |
| | | | (Pen G or V) - oral | pruritus, rash, shortness of | | swelling; erythema 16 | | |
| | | | route | breath, rapid heart rate | | hours later | | |
| F | 57 | White | Unknown Penicillin | Urticaria, shortness of | 2 | Urticaria, pruritus, | Diphenhydramine | Possibly |
| | | | product | breath | | erythematous rash >18 | | |
| | | | | | | hours, dermatitis | | |
| M | 54 | White | Unknown Penicillin | Angioedema | 1 | Flushing: Scalp flushing | None | Probably |
| | | | product-oral route | | | at 30 min | | |
| F | 66 | White | Unknown Penicillin | Urticaria | 1 | Rash pruritic: at 3 hours | Topical | Probably |
| | | | product-oral route | | | | hydrocortisone 1% | |
| F | 60 | White | Amoxicillin - oral | Urticaria, angioedema, | 1 | Macular rash and pruritus | Cetirizine | Probably |
| | | | route | pruritus, rash, nausea/ | | after 11 hours | | |
| | | | | vomiting | | | | |
| F | 27 | White | Benzylpenicillin | Pruritus, rash | 1 | Rash | None | Probably |
| | | | (Pen G or V) - oral | | | | | |
| | | | route | | | | | |

*AE event is coded using the MedDRA 16.0. Grade 1 - mild, Grade 2 - moderate, Grade 3 - severe, Grade 4 - life-threatening, Grade 5 - fatal as specified on the Adverse Event case-report form. There were no Serious Adverse Events (SAE).
†Determination of the adverse event as an IgE dependent immunological reaction and the relationship of the adverse event to the amoxicillin was determined by the enrolling investigator and the sponsor. If there were multiple IgE symptoms reported for the subject, the maximum AE grade and relationship code is reported in the table.

As shown in Table 4, all but one of the reactions was mild or moderate, and most patients were treated with antihistamines; one patient was treated with prednisone. All the patients recovered completely without sequalae. The NPV of the penicillin skin test kit in the PP population was 365/373, or 97.9% (96.4, 99.4 95% CI; p<0.0001). There were no significant demographic or clinical differences in these eight patients, compared to the 365 challenged subjects who did not react, with the exception of a higher prevalence of shortness of breath in historical penicillin reaction (Table 5).

TABLE 5
Demographics, asthma status and penicillin allergy history of penicillin history-positive
subjects with negative skin test by reaction to the oral challenge with Amoxicillin

| | | | ITT | | P† | Per Protocol | | P† |
|---|---|---|---|---|---|---|---|---|
| | Reacted* | | No Reaction | | React vs | No Reaction | | React vs |
| Characteristics at enrollment | (N = 8) | | (N = 383) | | ITT no react | (N = 365) | | PP no react |
| Demographics | | | | | | | | |
| Age (years), mean (SD) | 56.1 | (13.5) | 50.6 | (16.5) | 0.35 | 50.6 | (16.6) | 0.35 |
| [Minimum, maximum] | [27.6, 72.3] | | [18.4, 87.6] | | | [18.8, 87.6] | | |
| Female sex | 5 | (62%) | 281 | (73%) | 0.45 | 269 | (74%) | 0.44 |
| Ethnicity: Hispanic | 0 | (0%) | 12 | (3%) | 1.00 | 10 | (3%) | 1.00 |
| Race/ethnicity | | | | | 1.00 | | | 1.00 |
| Any Hispanic or Latino | 0 | (0%) | 12 | (3%) | | 10 | (3%) | |
| Non-Hispanic: | | | | | | | | |
| White | 8 | (100%) | 348 | (91%) | | 332 | (91%) | |
| Black | 0 | (0%) | 10 | (3%) | | 10 | (3%) | |
| Other‡ | 0 | (0%) | 13 | (3%) | | 13 | (4%) | |
| Asthma status | | | | | | | | |
| Active asthma | 4 | (50%) | 105 | (27%) | 0.23 | 98 | (27%) | 0.22 |
| Peak expiratory flow rate | 430.0 | (72.6) | 429.7 | (99.3) | 0.99 | 427.6 | (100.0) | 0.96 |
| (PEFR) (L/min), mean (SD)§ | | | | | | | | |
| Penicillin allergy history | | | | | | | | |
| Penicillin compounds | | | | | 1.00 | | | 1.00 |
| responsible for reaction¶ | | | | | | | | |
| Benzylpenicillin (Pen G or | 3 | (38%) | 127 | (33%) | | 122 | (33%) | |
| VK) | | | | | | | | |
| Amoxicillin | 2 | (25%) | 107 | (28%) | | 102 | (28%) | |
| Amoxicillin-clavulanic acid | 0 | (0%) | 15 | (4%) | | 15 | (4%) | |
| Other penicillin product | 0 | (0%) | 7 | (2%) | | 6 | (2%) | |
| Unknown penicillin product | 3 | (38%) | 127 | (33%) | | 120 | (33%) | |
| Allergy symptoms reported∥ | | | | | | | | |
| Uticaria | 5 | (62%) | 223 | (58%) | 1.00 | 220 | (60%) | 1.00 |
| Pruritus or flushing | 4 | (50%) | 212 | (55%) | 1.00 | 207 | (57%) | 0.73 |
| Maculopapular rash | 5 | (62%) | 168 | (44%) | 0.47 | 154 | (42%) | 0.29 |
| Angioedema | 4 | (50%) | 74 | (19%) | 0.05 | 73 | (20%) | 0.06 |
| Shortness of breath | 3 | (38%) | 41 | (11%) | 0.05 | 37 | (10%) | 0.04 |
| Lightheadedness or loss of | 1 | (12%) | 28 | (7%) | 0.46 | 26 | (7%) | 0.46 |
| consciousness | | | | | | | | |
| Nausea or vomiting or | 1 | (12%) | 20 | (5%) | 0.36 | 19 | (5%) | 0.36 |
| abdominal pain | | | | | | | | |
| Rapid heart rate | 2 | (25%) | 21 | (5%) | 0.07 | 19 | (5%) | 0.06 |
| Fever | 1 | (12%) | 17 | (4%) | 0.32 | 16 | (4%) | 0.31 |
| Diarrhea | 0 | (0%) | 6 | (2%) | 1.00 | 5 | (1%) | 1.00 |
| Joint pain | 0 | (0%) | 5 | (1%) | 1.00 | 3 | (1%) | 1.00 |
| Treatments for reaction** | | | | | | | | |
| None | 2 | (25%) | 93 | (24%) | 1.00 | 89 | (24%) | 1.00 |
| Antihistamine | 0 | (0%) | 94 | (25%) | 0.21 | 92 | (25%) | 0.21 |
| Epinephrine | 0 | (0%) | 16 | (4%) | 1.00 | 13 | (4%) | 1.00 |
| Corticosteroids | 1 | (12%) | 30 | (8%) | 0.49 | 27 | (7%) | 0.47 |
| Other | 0 | (0%) | 33 | (9%) | 1.00 | 31 | (8%) | 1.00 |
| Cannot recall | 5 | (62%) | 154 | (40%) | 0.28 | 145 | (40%) | 0.28 |
| Reaction observed by provider | | | | | 0.48 | | | 0.47 |
| Yes | 7 | (88%) | 258 | (67%) | | 251 | (69%) | |
| No | 0 | (0%) | 67 | (17%) | | 63 | (17%) | |
| Unknown | 1 | (12%) | 58 | (15%) | | 51 | (14%) | |
| Assessment of IgE-mediated | | | | | 0.24 | | | 0.19 |
| penicillin allergy by history | | | | | | | | |
| Unlikely | 0 | (0%) | 5 | (1%) | | 0 | (0%) | |
| Possible | 2 | (25%) | 158 | (41%) | | 150 | (41%) | |
| Probable | 4 | (50%) | 191 | (50%) | | 186 | (51%) | |
| Definite | 2 | (25%) | 29 | (8%) | | 29 | (8%) | |

*Reacted denotes an IgE-mediated Adverse Event following the oral amoxicillin challenge; no reaction denotes no IgE adverse event reported following the challenge.
No. (percent) reported for categorical characteristics; mean (standard deviation) reported for continuous characteristics.
†P determined from Fisher's exact test for categorical variables, and two-sample t-test for continuous variables.
‡Other race for ITT-no reaction and per-protocol (PP)-no reaction includes: 2 subjects are American Indian, 5 Asian, 1 Hawaiian/South Pacific Islander, 5 Other including mixed.
§Peak expiratory flow rate is only collected for active asthma subject.
¶Penicillin compound reported for the subject's first reported reaction.
For ITT: 15/391 (4%) subjects in ITT population reported 2 penicillin reactions in the history [0/8 (0%) Reacted vs 15/383 (4%) no reaction; P = 1.00)]; in the PP population, 12 (3%) subjects reported 2 penicillin reactions [0/8 (0%) Reacted vs 12/365 (3%) no reaction; P = 1.00)].
∥Subjects may report more than one symptom for the reaction.
** Subjects may report more than one treatment for the reaction.
Other treatments for reaction included: lotions, creams, ER/hospitalization, oatmeal bath, oxygen.

### DISCUSSION

The penicillin skin test kit of the present invention, consisting of benzylpenicilloyl polylysine (PRE-PEN®), MDM (penicillin G potassium, penicilloic acid, and penilloic acid) and amoxicillin sodium, demonstrated very high NPV of 97.9% in a large group of patients with convincing histories of IgE-dependent penicillin allergy. The fact that only 8 out of a total of 365 skin test-negative subjects (PP population) with a history of penicillin allergy developed an IgE-dependent allergic-type reaction, all but one mild to moderate in degree, to an oral challenge with amoxicillin underscores the utility of the diagnostic kit. The NPV is comparable to the published literature involving similar populations and single challenge dose (Fox S, Park M. J Allergy Clin Immunol Pract. (2014) 2:439-44; Gadde J, Spence M, Wheeler B, Adkinson N F. JAMA. (1993) 270:2456-63; Macy E, Ngor E W. J Allergy Clin Immunol Pract. (2013) 1:258-63; Sogn D D, Evans R, Shepherd G M, Casale T B, Condemi J, Greenberger P A, et al. Arch Intern Med. (1992) 152:1025-32). The positive predictive value (PPV) of the penicillin skin test kit was not determined. Based on limited number of penicillin challenges of skin test-positive individuals in the published literature, the PPV of penicillin skin testing is approximately 50% (Adkinson N F Jr, Thompson W L, Maddrey W C, Lichtenstein L M. N Engl J Med. (1971) 285:22-24; Green G R, Rosenblum A H, Sweet L C. J Allergy Clin Immunol. (1977) 60:339-45; Sogn D D, Evans R, Shepherd G M, Casale T B, Condemi J, Greenberger P A, et al. Arch Intern Med. (1992) 152:1025-32; Solley G O, Gleich G J, Dellen R G V. J Allergy Clin Immunol. (1982) 69:238-44) which is comparable to food and hymenoptera skin testing. Furthermore, since the rate of positive tests is low (14% in this study), the primary usefulness of penicillin skin testing is in ruling out rather than confirming the allergy. A negative penicillin skin test result changes patients' management in that it opens up the potential use of beta-lactam antibiotics, whereas a positive test results in continued avoidance of those antibiotics. Therefore, determination of the exact PPV (i.e., whether it is 30% or 80%) has little clinical utility.

The penicillin skin test kit of the present invention is an improvement over skin test reagents that are available presently in the US. The kit of the present invention includes the more important and well recognized penicillin skin test allergens in the published literature, synthesized and standardized according to FDA requirements. For efficiency and clinical application, the minor determinants in the kit, penicillin G potassium, penicilloic acid and penilloic acid, were combined into one container (MDM) rather than provided separately.

At the present time, the only penicillin skin test reagent that has received FDA approval is benzylpenicilloyl polylysine (sold under the tradename PRE-PEN®), and that was in 1974. Other reagents for skin testing, such as penicillin G, have been utilized individually off-label or, in the case of penicilloate and penilloate, have not been used at all. The additional component of the minor allergenic determinants penicilloate, penilloate and amoxicillin in penicillin skin testing, versus testing with only PRE-PEN® and off-label penicillin G, increases the confidence in the overall determination because these components identify a sub-population of patients who test positive only to these reagents. In this study, 65.6% of the skin test positive subjects were positive only to MDM alone, amoxicillin sodium alone, or both MDM and amoxicillin. Consequently, skin testing with only PRE-PEN® and penicillin G potassium likely would not have detected many of these individuals. In fact, in large-scale studies in which individual minor determinant skin test reagents rather than a tripartite MDM were utilized, about 10% of the penicillin skin test-positive patients were consistently positive to only penicilloate and/or penilloate, and negative to both PRE-PEN® and penicillin G (Fox S, Park M. J Allergy Clin Immunol Pract. (2014) 2:439-44; Macy E, Burchette R. Allergy. (2002) 57:1151-8; Macy E, Richter P K, Falkoff R, Zeiger R. J Allergy Clin Immunol. (1997) 100:586-91; Matheu V, Perez E, Gonzalez R, Poza P, de la Torre F, Sanchez-Machin I, et al. J Invest Allergol Clin Immunol. (2007) 17:257-60; Park M, Matesic D, Markus P J, Li J T. Ann Allergy Asthma Immunol. (2007) 99:54-8; Sullivan et al. J. Allergy Clin. Immunol. (1981) 68:171-180). Thus, it is highly likely that use of the tripartite MDM will increase the number of penicillin sensitized patients identified, compared with skin testing with only penicillin G potassium.

6.3% (4/64) of skin test-positive patients were positive only to amoxicillin. This result is comparable to rates in published US studies, which have ranged from 3.1% to 6% (Fox S, Park M. J Allergy Clin Immunol Pract. (2014) 2:439-44; Lin E, Saxon A, Riedl M. Int Arch Allergy Immunol. (2010) 152:313-8; Park M, Matesic D, Markus P J, Li J T. Ann Allergy Asthma Immunol. (2007) 99:54-8). In contrast, research from Europe has shown much higher rates of selective amoxicillin allergy on skin testing, generally 25-50% (Bousquet P J, Co-Minh H B, Arnoux B, Daures J P, Demoly P. J Allergy Clin Immunol. (2005) 115:1314-6; Matheu V, Perez E, Gonzalez R, Poza P, de la Torre F, Sanchez-Machin I, et al. J Invest Allergol Clin Immunol. (2007) 17:257-60; Romano A, Bousquet-Rouanet L, Viola M, Gaeta F, Demoly P, Bousquet P J. Allergy. (2009) 64:249-53). The reason for these geographical differences is unknown, but underscore the importance of including amoxicillin in a penicillin skin test kit.

### Example 2

The following example illustrates preparation and use of a kit of the present invention.

### Materials for Use in the Kit

### (1) Package Insert

### (2) Instructions for Use

### (3) 1 Vial MDM for Injection

### (4) 1 Vial Diluent for MDM

### (5) 1 Vial Amoxicillin Sodium for Injection

### (6) 1 Vial Diluent for Amoxicillin Sodium

(7) 1 Ampoule benzylpenicilloyl polylysine (PRE-PEN®)

### (8) 1 Vial Positive Skin Test Control e.g., Histamine (Histatrol)

### (9) 1 Histatrol Package Insert

(10) 1 Vial Negative Skin Test Control, e.g., Saline (0.9% sodium chloride)
Additional items optionally contained in the kit include:

### (11) Skin Test Applicators

### (12) Puncture Skin Test Location Template

### (13) Intradermal Test Location Template

### (14) Alcohol Swabs

### (15) Syringe Labels

### (16) Skin Test Ruler

### (17) Disposable Syringes

### (18) Injectable Epinephrine

(19) Pen for skin markings
(20) Work tray

### Test Information, Precautions, and Storage

The skin test reagents in the kit of the invention should be reconstituted and administered by a healthcare provider with expertise in allergy skin testing for allergic diseases. In line with good clinical practice, monitoring of patients after administration of skin test reagents is recommended. In addition, the following precautions and preliminary steps should be taken:

(A) Hands should be washed before and after performing procedures.

(B) Gloves should be worn during performance of testing.

(C) The contents of the kit must be kept refrigerated at 2-8° C. until use.

(D) The vial of reconstituted MDM for Injection and Amoxicillin Sodium for Injection should be used within 90 minutes of reconstitution with appropriate diluents.

### Reconstitution of MDM

Before using MDM for Injection, the lyophilized powder must be reconstituted by mixing with the Diluent for MDM. The following are sequential steps for reconstitution of the MDM.

1. Take 1 vial of MDM for Injection and place on counter. The powder in the vial should look like a white to off-white tablet that is whole or in pieces.

2. Take 1 vial of Diluent for MDM and place on counter. The liquid should be clear, colorless, and free of visible particulates.

3. Remove the protective caps from the tops of both vials.

4. Clean the rubber stopper on the top of both vials with an alcohol swab.

5. Take one of the syringes and remove the protective cap.

6. Fill the syringe with air by pulling back on the plunger to 0.4 mL. Hold the vial upright. Do not touch the cleaned top of the vial with your hands.

7. Push the needle through the center of the rubber stopper of the vial. Slowly inject all the air from the syringe into the air space above the diluent in the vial.

8. Turn the vial upside down and withdraw only 0.4 mL of diluent.

9. With the needle still inserted in the vial, check the syringe for air bubbles. If there are any air bubbles, gently tap the syringe with your finger until the air bubbles rise to the top of the syringe. Slowly push the plunger up to remove the air bubbles. If you push diluent back into the vial, slowly pull back on the plunger to draw the correct 0.4 mL amount of diluent back into the syringe.

10. Insert the needle through the center of the rubber stopper of the MDM for Injection vial. Due to stopper design it is important to push through the center of the rubber stopper to avoid bending or breaking the needle. Do not touch the cleaned rubber stopper.

11. Place the needle tip, at an angle, against the side of the vial. Slowly push the plunger down to inject the 0.4 mL diluent. The stream of diluent should run down the side of the vial. To prevent bubbles from forming, do not aim the stream of diluent directly on the medicine in the bottom of the vial. The syringe should not be utilized again.

12. Gently swirl the vial in a circular motion, until the MDM powder is completely dissolved (mixed together).
a. Do not shake the vial as this may lead to product foaming or precipitation. If any powder remains undissolved in the vial, gently turn the vial upside down until all of the powder is dissolved.
b. The solution may look cloudy or bubbly for a few minutes. If air bubbles form, wait until the solution settles and all bubbles rise to the top.
c. Visually inspect the reconstituted solution for particulate matter and clarity before use. After the MDM powder completely dissolves, the solution should be clear, colorless and without particles. It is normal to see a ring of foam or bubbles on the surface. Do not use the mixed solution if you see particles in it, or it is not clear and colorless.

13. After the MDM powder completely dissolves, clean the rubber stopper again with an alcohol swab and place on a clean, well-lit, flat work surface.

14. The reconstituted MDM for Injection should be used within 90 minutes.

### Reconstitution of Amoxicillin Sodium for Injection

Before using Amoxicillin Sodium for Injection, the amoxicillin powder must be reconstituted by mixing with 1.0 mL of Diluent for Amoxicillin Sodium. The following are sequential steps for the reconstitution of the Amoxicillin:

1. Take 1 vial of Amoxicillin Sodium for Injection and place on counter. The powder in the vial should look like a pale yellow to tan powder.

2. Take 1 vial of Diluent for Amoxicillin Sodium and place on counter. The liquid should be clear, colorless, and free of visible particulates.

3. Remove the protective caps from the tops of both vials.

4. Clean the rubber stopper on the top of both vials with an alcohol swab.

5. Take one unused syringe and remove the protective cap.

6. Fill the syringe with air by pulling back on the plunger to 1.0 mL. Hold the vial upright. Do not touch the cleaned top of the vial with your hands.

7. Push the needle through the center of the rubber stopper of the vial. Slowly inject all the air from the syringe into the air space above the diluent in the vial.

8. Turn the vial upside down and withdraw only 1.0 mL of diluent.

9. With the needle still inserted in the vial, check the syringe for air bubbles. If there are any air bubbles, gently tap the syringe with your finger until the air bubbles rise to the top of the syringe. Slowly push the plunger up to remove the air bubbles. If you push diluent back into the vial, slowly pull back on the plunger to draw the correct amount of diluent back into the syringe.

10. Insert the needle through the center of the rubber stopper of the Amoxicillin Sodium for Injection vial. Do not touch the cleaned rubber stopper.

11. Place the needle tip, at an angle, against the side of the vial. Slowly push the plunger down to inject the 1.0 mL of diluent. The stream of diluent should run down the side of the vial. To prevent bubbles from forming, do not aim the stream of diluent directly on the medicine in the bottom of the vial. The syringe with attached needle that you have just used should not be utilized again.

12. Gently swirl the vial in a gentle circular motion, until the Amoxicillin powder is completely dissolved (mixed together).
a. Do not shake the vial as this may lead to product foaming or precipitation. If any powder remains undissolved in the vial, gently turn the vial upside down until all of the powder is dissolved.
b. The solution may look cloudy or bubbly for a few minutes. If air bubbles form, wait until the solution settles and all bubbles rise to the top.
c. Visually inspect the reconstituted solution for particulate matter and clarity before use. After the Amoxicillin powder completely dissolves, the solution should be clear, colorless to slightly yellow and free of visible particles. It is normal to see a ring of foam or bubbles on the surface. Do not use the mixed solution if you see particles in it, or it is not clear and colorless.

13. After the Amoxicillin powder completely dissolves, clean the rubber stopper again with an alcohol swab and place on a clean, well-lit, flat work surface.

14. The reconstituted Amoxicillin Sodium for Injection should be used within 90 minutes.

Following preparation of the above reagents, puncture (epicutaneous) and intradermal testing follows. Benzylpenicilloyl polylysine, MDM and amoxicillin are first administered on patient by skin puncture (epicutaneous) testing (described below) before proceeding to intradermal testing. Intradermal testing should only be performed if all skin puncture testing to reagents are found to be NEGATIVE and there is a POSITIVE reaction to the Positive Skin Test Control (Histamine).

### Puncture Testing

1. Puncture tests are performed at marked sites simultaneously for Histamine, Saline, benzylpenicilloyl polylysine, MDM, and amoxicillin.

2. Prior to performing the testing it is recommended that syringes containing each reagent are labeled appropriately to avoid product mix-up.

3. Draw up 0.2 mL-0.3 mL of each appropriate solution and place them on a clean, well-lit, flat work surface.

4. The Positive Skin Test Control—Histamine may not need to be syringed as it may be provided in a vial with a black dropper.

5. Testing must be initiated within 90 minutes of reconstitution of MDM and Amoxicillin and performed on the inner right forearm (unless there are anatomical constraints).

6. Clean the skin surface of the inner right forearm with an alcohol wipe and let dry.

7. Mark the test sites to ensure accurate identification and placement of products. A Puncture Skin Test Location Template is provided as a tool for this purpose. It is recommended that a fine point ink pen is used to mark the skin using the Template provided.

8. Using the Positive Skin Test Control-Histamine, and after locating the test sites, apply a small drop of each test solution just lateral to the corresponding pre-marked site on the testing arm. The needle tip and dropper should not touch the skin.

9. After the drop is placed, the labeled syringe should be placed back on the flat clean surface for subsequent intradermal testing.

10. After each skin test solution drop is placed on the skin, use the applicator using the rotation technique while holding the gripping area between index finger and thumb. Press points vertically against skin with enough pressure to slightly indent skin. Maintain pressure on skin while rotating shaft clockwise or counter-clockwise. Use a separate sterile applicator to puncture each drop of test solution. Do not draw blood.

11. Observe the area in 15-20 minutes for the appearance of a wheal, erythema, and the occurrence of itching at the test site.

12. Immediately prior to recording results use a pen to mark the edge of the wheal and the surrounding erythema. A ruler is provided as a tool to assist with measurement of wheal and flare. Using the provided ruler, measure and record the longest diameter for both wheal and (if possible) erythema.

13. A positive puncture test reaction consists of the development within 10-20 minutes of a pale wheal, sometimes with an irregular outline (“pseudopods”), surrounding the puncture site and measuring in its longest diameter ≥5 mm. A larger, irregular circle of erythema usually surrounds the wheal in a positive puncture test, but is not considered in defining positivity.

14. As soon as a positive response as defined in Step 13 is clearly evident, the solution over the puncture should be immediately blotted off.

15. DO NOT Proceed to Intradermal Testing and Stop Testing if any of the following occur.
a. If there is a positive reaction to benzylpenicilloyl polylysine, MDM, and/or amoxicillin. Proceeding to intradermal testing is unnecessary at this point since it would not affect the clinical interpretation.
b. In the event Saline has a positive response (dermatographism demonstrated) do not proceed to intradermal testing of any reagent.
c. Do not move forward if the puncture skin test response to the histamine is negative (patient suppressed).

16. A negative or equivocal puncture test reaction consists of a wheal <5 mm in longest diameter, usually with little or no surrounding erythema and no itching.
a. An intradermal test may then be performed for all reagents if the skin puncture tests to benzylpenicilloyl polylysine, MDM, amoxicillin, and saline are all negative or equivocal and the response to Histamine is positive.

### Intradermal (ID) Testing

The test solutions for intradermal testing include benzylpenicilloyl polylysine, MDM, Amoxicillin, and a Negative Skin Test Control (Saline).

1. Intradermal testing will usually be performed on the left arm.

2. Prepare with an alcohol swab the LEFT FOREARM, or alternative site if justified.

3. Mark the test sites to ensure accurate identification and placement of products. An Intradermal Test Location Template is provided as a tool for this purpose. It is recommended that a pen be used to mark the skin using the Template provided. Duplicate intradermal skin testing of each product is recommended.

4. It is recommended that the labeled syringes that contain the skin test solutions which were previously drawn into these syringes for puncture testing be used to perform the intradermal tests.

5. Perform intradermal testing.

6. Just lateral to the corresponding marked skin test site, insert the needle, bevel up, immediately below the skin surface.

7. Inject an amount of test solution sufficient to raise a small intradermal bleb of about 3 mm in diameter.

8. Perform duplicate testing—It is recommended to inject a duplicate test just lateral to the marked second site about 2 cm apart. This is useful for evaluating equivocal reactions.

9. It is recommended that a pen be used to define the circumference of the initial wheal immediately after each pair of intradermal skin tests are placed in order to subsequently determine if the wheal has increased in size.

10. Observe for Reaction—Most skin reactions will develop within 5-15 minutes and the response to the skin test is read at 20 minutes as follows:
a. Negative response—no increase in size of original bleb and no greater reaction than the Saline test site.
b. Positive response—itching and significant increase in size of original blebs to at least 5 mm. Wheal may exceed 20 mm in diameter and exhibit pseudopods.
c. Ambiguous (Equivocal) response—wheal only slightly larger than initial injection bleb, with or without accompanying erythematous flare and slightly larger than the control site; OR discordance between duplicates.
d. Positive response to Saline—If the control site demonstrates a wheal greater than 2-3 mm, repeat the test, and if the same reaction is observed dermatographism has been demonstrated.

11. Prior to recording the results it is recommended to again use pen to mark the edge of the wheal (if it has increased in size) and the surrounding erythema. Using the provided ruler, measure and record the longest diameter for both wheal and (if possible) erythema.

12. Recommended procedure for ambiguous (equivocal) test
a. If response to any test is considered ambiguous (equivocal, unable to decide OR discordant duplicates) repeating that skin test in duplicate together with a negative control in duplicate is recommended. Usually the true result will be reflected in agreement among 3 of the 4 replicates.

At the completion of testing, all kit contents and materials used for skin testing should be disposed of appropriately in accordance with state and federal regulatory guidelines.

This disclosure further encompasses the following aspects.

Aspect 1. A kit for evaluating on the skin of a patient the sensitivity to penicillin, comprising:

(A) a first vial containing a major determinant mixture, said major determinant mixture comprising benzylpenicilloyl polylysine;

(B) a second vial containing lyophilized minor determinant mixture, said minor determinant mixture comprising a lyophilized mixture of neutralized:
(1) penicillin G potassium;
(2) penicilloic acid; and
(3) penilloic acid;

(C) a third vial containing amoxicillin sodium; and

(D) instructions for carrying out a method to evaluate the sensitivity to penicillin on the skin of a patient.

Aspect 2. The kit of aspect 1, wherein said benzylpenicilloyl polylysine has the structure of Formula I:

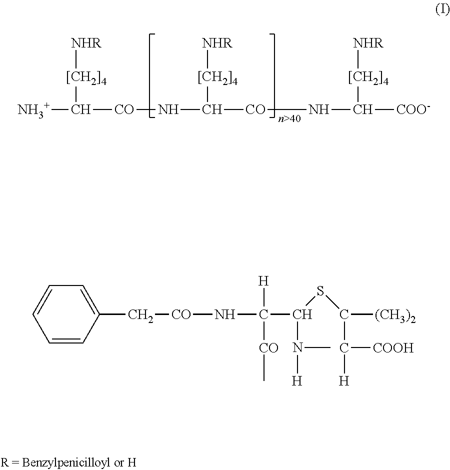

Aspect 3. The kit of aspect 1, wherein said benzylpenicilloyl polylysine is in the form of a sterile solution.
Aspect 4. The kit of aspect 3, wherein said first vial contains said benzylpenicilloyl polylysine at a concentration ranging from about 1×10⁻⁵ M to about 10×10⁻⁵ M.
Aspect 5. The kit of aspect 3, wherein said first vial contains said benzylpenicilloyl polylysine at a concentration of approximately 6×10⁻⁵ M.
Aspect 6. The kit of aspect 1, wherein said minor determinant mixture is in the form of a sterile powder.
Aspect 7. The kit of aspect 1, wherein said second vial contains said benzylpenilloic acid, said benzylpenicillin, and said benzylpenicilloic acid in amounts ranging from 0.01 to 10 mg for each component.
Aspect 8. The kit of aspect 1, wherein said second vial contains said benzylpenilloic acid, said benzylpenicillin, and said benzylpenicilloic acid in amounts ranging from 0.01 to 10 mg for each component.
Aspect 9. The kit of aspect 1, wherein each component of said minor determinant mixture is present in a molar ratio of 1:1:1.
Aspect 10. The kit of aspect 1, where said minor determinant mixture has a moisture content of about 1 to 1.5%.
Aspect 11. The kit of aspect 1, wherein said second vial further comprises a buffer.
Aspect 12. The kit of aspect 1, wherein said third vial contains from 1 to 50 mg of said amoxicillin sodium.
Aspect 13. The kit of aspect 1, wherein said amoxicillin sodium has the structure of Formula (V):

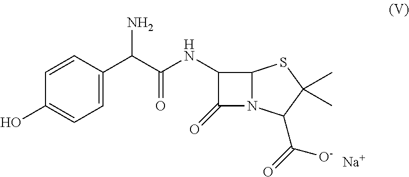 [SMILES: CC1(C)SC2C(NC(=O)C(N)c3ccc(O)cc3)C(=O)N2C1C(=O)[O-] ; [Na+]]

Aspect 14. The kit of aspect 1, further comprising one or more positive or negative controls.
Aspect 15. The kit of aspect 1, further comprising one or more additional components selected from the group consisting of skin test applicators, test templates, syringe labels, skin test rulers, and alcohol wipes.
Aspect 16. A method of evaluating on the skin of a patient sensitivity to penicillin using the kit of aspect 1.

Any publications or references mentioned in this specification are indicative of the level of those skilled in the art to which the invention pertains. All patents, publications, and/or references herein are incorporated by reference to the same extent as if each individual publication was specifically and individually indicated as having been incorporated by reference in its entirety.

What is claimed is:

1. A kit for evaluating on the skin of a patient the sensitivity to penicillin, comprising:
(A) a first vial containing a major determinant mixture, said major determinant mixture comprising benzylpenicilloyl polylysine;
(B) a second vial containing lyophilized minor determinant mixture, said minor determinant mixture comprising a lyophilized mixture of neutralized:
(1) penicillin G potassium;
(2) penicilloic acid; and
(3) penilloic acid;
(C) a third vial containing amoxicillin sodium; and
(D) instructions for carrying out a method to evaluate the sensitivity to penicillin on the skin of a patient.

2. The kit of claim 1, wherein said benzylpenicilloyl polylysine has the structure of Formula I:

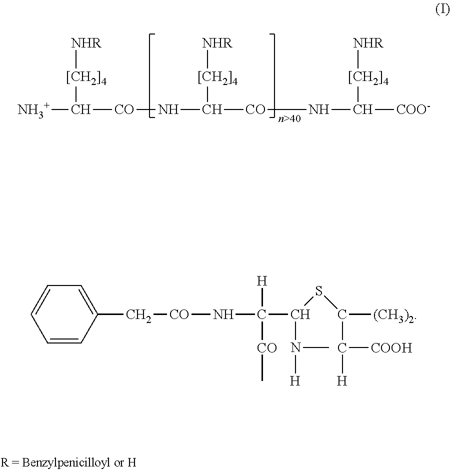

3. The kit of claim 1, wherein said benzylpenicilloyl polylysine is in the form of a sterile solution.

4. The kit of claim 3, wherein said first vial contains said benzylpenicilloyl polylysine at a concentration ranging from about 1×10⁻⁵ M to about 10×10⁻⁵ M.

5. The kit of claim 3, wherein said first vial contains said benzylpenicilloyl polylysine at a concentration of approximately 6×10⁻⁵ M.

6. The kit of claim 1, wherein said minor determinant mixture is in the form of a sterile powder.

7. The kit of claim 1, wherein said second vial contains said benzylpenilloic acid, said benzylpenicillin, and said benzylpenicilloic acid in amounts ranging from 0.01 to 10 mg for each component.

8. The kit of claim 1, wherein said second vial contains said benzylpenilloic acid, said benzylpenicillin, and said benzylpenicilloic acid in amounts ranging from 0.01 to 10 mg for each component.

9. The kit of claim 1, wherein each component of said minor determinant mixture is present in a molar ratio of 1:1:1.

10. The kit of claim 1, where said minor determinant mixture has a moisture content of about 1 to 1.5%.

11. The kit of claim 1, wherein said second vial further comprises a buffer.

12. The kit of claim 1, wherein said third vial contains from 1 to 50 mg of said amoxicillin sodium.

13. The kit of claim 1, wherein said amoxicillin sodium has the structure of Formula (V):

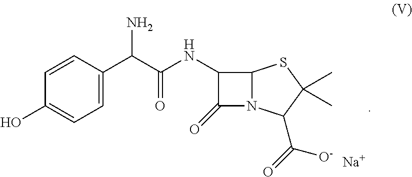 [SMILES: CC1(C)SC2C(NC(=O)C(N)c3ccc(O)cc3)C(=O)N2C1C(=O)[O-] ; [Na+]]

14. The kit of claim 1, further comprising one or more positive or negative controls.

15. The kit of claim 1, further comprising one or more additional components selected from the group consisting of skin test applicators, test templates, syringe labels, skin test rulers, and alcohol wipes.

16. A method of evaluating on the skin of a patient sensitivity to penicillin using the kit of claim 1.
